(19) United States
(12) Patent Application Publication
(10) Pub. No.: US 2009/0111108 A1
(43) Pub. Date: Apr. 30, 2009

(54) Method of Detecting Genetic Mutations

(76) Inventors: Feng Gao, Durham, NC (US); Jun Zhu, Durham, NC (US)

Correspondence Address: NIXON & VANDERHYE, PC, 901 NORTH GLEBE ROAD, 11TH FLOOR, ARLINGTON, VA, 22203, US

(21) Appl. No.: 12/226,139

(22) PCT Filed: Apr. 10, 2007
(86) PCT No.: PCT/US2007/008857

§ 371 Date: Dec. 18, 2008

Related U.S. Application Data
(60) Provisional application No. 60/790,535, filed on Apr. 10, 2006.
(60) Provisional application No. 60/878,700, filed on Jan. 5, 2007.

(51) Int. Cl. C12Q 1/68

(52) U.S. Cl. 435/6

(57) ABSTRACT

The present invention relates, in general, to drug resistance, and, in particular, to a method of detecting drug resistance populations, including minor drug resistance viral populations.

This application claims priority from U.S. Provisional Application No. 60/790,535, filed Apr. 10, 2006 and from U.S. Provisional Application No. 60/878,700, filed Jan. 5, 2007, the entire contents of both applications being incorporated herein by reference.

This invention was made with government support under RO1 GM065057 awarded by the National Institutes of Health. The government has certain rights in the invention.

### TECHNICAL FIELD

The present invention relates to a method of detecting genetic variants, including genetic variants associated with drug resistance, cancer and genetic dysfunctional diseases.

### BACKGROUND

Combinational drug therapy or highly active antiretroviral therapy (HAART) is the primary means to treat HIV-infected individuals. Since HAART can often suppress viral replication to an undetectable level in patient blood, the morbidity and mortality of HIV infected patients have decreased dramatically (Egger et al, BMJ 315:1194-9 (1997), Palella et al, N Engl J Med 338:853-60 (1998)). However, responses to HAART are often variable. Some patients respond well with successful suppression of viral replication, but others fail the treatment soon after HAART. Analyses of protease and reverse transcriptase (RT) gene sequences from patients who fail HAART show resistance to multiple drugs (Condra et al, J Virol 70:8270-6 (1996), Larder and Kemp, Science 246:1155-8 (1989), Johnson et al, Top. HIV Med. 13:125-131 (2005)). The biological functions of these mutations have been confirmed with in vitro assays by introducing these mutations into infectious molecular clones and testing their susceptibility to drugs (Kellam and Larder, J Virol 69:669-74 (1995)). These data suggest that genetic changes are the foundation for multiple-drug resistance (MDR).

HIV-1 genomes are highly variable and each infected individual harbors a multitude of genetically similar but different viral variants (quasispecies). When drug resistance occurs in treated patients, it has been shown that at least certain of the viral genomes do not contain drug-resistance mutations. In viruses that contain resistance mutations, the number of such mutations per genome varies. Therefore, the relationship between drug resistance mutations and treatment outcome can only be fully understood by determining the identity, nature and number of drug resistance mutations in each viral genome from the large viral population in an infected individual. Understanding mechanisms of drug resistance will allow for the development of more effective antiretroviral agents, better treatment regimens, and more accurate prediction of treatment efficacy.

Two methods are currently used to detect the presence of drug-resistance mutations and to predict the possibility of resistance to a new HAART regimen (Petropoulos et al, Antimicrob Agents Chemother 44:920-8 (2000), Van Laethem et al, J Acquir Immune Defic Syndr 22:107-18 (1999)). However, both assays have a low sensitivity for detecting minor drug-resistant viral populations and cannot reliably determine of the percentage of drug resistance viruses among wild type viruses. Furthermore, because drug-resistance mutations are detected on the basis of viral population, neither assay is suitable for linkage analysis, which can reveal critical information required for understanding drug resistance mechanisms and prediction of treatment outcomes. Recently, a variety of real-time PCR methods have been developed to detect minor drug-resistant populations (Charpentier et al, J Virol 78:4234-47 (2004), Hance et al, J Virol 75:6410-7 (2001), Metzner et al, J Infect Dis 188:1433-43 (2003), Metzner et al, Aids 19:1819-25 (2005), Mohey et al, J Clin Virol 34:257-67 (2005), Thelwell et al, Nucleic Acids Res 28:3752-61 (2000), Whitcombe et al, Nat Biotechnol 17:804-7 (1999)). These assays, however, can only detect one drug resistance mutation at a time, which makes them less practical for analyzing all drug-resistance mutations. Furthermore, given the high genetic variability in HIV-1 genomes, it would be a daunting task to design primer sets that could be used to detect all drug resistance mutations for most genetic variants.

Clonal sequencing has been used to study minor drug resistance populations, drug resistance mutation distribution on each viral genome, and linkage analysis from a large number of viral clones. Although the sequences of the whole amplicons can be obtained, this method is labor intensive and time consuming. The sensitivity of the assay is limited by the number of clones available for analysis and the method is affected by recombination between different viral genomes or viral genome amplicons and resampling of the same molecules during PCR amplification. Limiting-dilution PCR or single genome amplification (SGA) can significantly decrease the possibility of the above concerns, but the high cost prevents its use for routine detection of resistance mutations (Palmer et al, J Clin Microbiol 43:406-13 (2005)).

High-throughput sequencing methods have been developed and can potentially be used to detect drug resistance mutations. Picoliter-scale and multiplex polony sequencing methods can be used to sequence viral genomes in a cost-effective manner (Margulies et al, Nature 437:376-80 (2005), Shendure et al, Science 309:1728-32 (2005)). The viral genomes are assembled from short DNA fragments (200 bp and 20 bp, respectively). Therefore, all resistance mutations cannot be obtained from one viral genome and the linkage analysis cannot be performed with sequences obtained through either method. Due to the high level of genetic variation, it would be difficult to design multiple primer sets to cover the pol gene that would contain all drug resistance mutations. Sample preparation for small numbers of RNA molecules from patient plasma is complicated in both methods (Rogers et al, Nature 437:326-7 (2005)).

In view of the above, there is a urgent need to develop methods that allow for detection of minor drug resistant populations (≦0.01%), simultaneous analysis of tens of thousands of viral genomes, identification of all primary mutations at the single molecule level using limited samples, determination of the percentage of different drug resistance mutations present in a viral genome, and linkage analysis. The present invention relates to a novel parallel allele-specific sequencing (PASS) assay that meets all above requirements. It can be used to study the mechanism of multiple drug resistance and it provides a method for predicting of treatment outcomes.

### SUMMARY OF THE INVENTION

The present invention relates to a method of detecting genetic variants, including genetic variants associated with drug resistance, cancer and genetic dysfunctional diseases. In a specific embodiment, the invention relates to an assay that can be used to detect minor drug resistance populations, e.g. viral populations.

Objects and advantages of the present invention will be clear from the description that follows.

### BRIEF DESCRIPTION OF THE DRAWINGS

FIGS. 1A and 1B. Schematic presentation of the parallel allele-specific sequencing (PASS) assay. (FIG. 1A) Unmodified 5′ primers, acrydited 3′ primers, DNA templates and PCR reagents were embedded in 6% acrylamide gel. Because the acrydited primers were covalently coupled with the polyacrylamide gel during polymerization and became immobilized, the amplified PCR products accumulated around the DNA templates and formed individual spots (polonies) at the amplification sites. After the double strand DNA was denatured, the free strand of DNA was washed off while the DNA strands extended from the acrydited primers were kept in the gel. The sequencing primers were then annealed to the single strand templates and extended with fluorescent-labeled wild type or resistance bases. Gels were then scanned on the Microarray Scanner to acquire images. (FIG. 1B) The 3′ end of the primer was juxtaposed to the drug-resistance mutation position. When the primers are extended with fluorescence-labeled nucleotides, the base-identity at the mutation site could be determined by the fluorophore incorporated. The template sequence is shown at the bottom with the M184V primer sequence underlined. The primer sequence is shown at the top. The added WT base is labeled with Cy3 and shown in green. The incorporated mutation base is labeled with Cy5 and shown in red.

FIGS. 2A-2C. Detection of minor drug-resistance mutations by PASS. Wild type (WT) and mutant clones were mixed together in a standard PASS assay. The polonies were sequenced with the M184V primer using Cy3-dATP for WT bases and Cy5-dCTP for mutations. The images were first acquired for the Cy3 WT channel (FIG. 2A) and then the Cy5 mutant channel (FIG. 2B). Finally, both were superimposed to each other to show mutant and WT viral populations in the same image (FIG. 2C).

FIGS. 3A-3C. Detection of minor drug-resistance populations by PASS. (FIG. 3A) The E44D mutant clone (WEAU-E44D) was mixed with WT clone (WEAU-wt) at 1:10 serial dilutions. After PCR amplification, the polonies were sequenced with the E44D primer using Cy3-dATP for the WT base (green) and Cy5-dCTP for the mutation (red). The molecule number of the 1:1,000 mutant/WT mixture as shown in FIG. 3B was increased by ten fold to better detect rare resistance mutations among a larger WT viral population. After PCR amplification, the polonies were sequenced with the E44D primer using Cy3-dATP for WT bases and Cy5-dCTP for mutations. Sensitivity of the PASS assay for detection of minor drug-resistance populations (FIG. 3C). The same experiments shown in FIG. 3A were repeated six times for each WT:mutatant ratio. The linear regression analysis were performed and plotted. The error bars stand for mean±SD.

FIGS. 4A-4C. Detection of multiple drug-resistance mutations on different molecules. Three drug-resistance mutations (L90M, E44D and M184V) were introduced into the WT WEAU pol gene. The WT clone (WEAU-wt) and three drug-resistance clones (WEAU-E44D, WEAU-L90M and WEAU-M184V) were mixed at a 1:1:1:1 ratio in a PASS assay. The polonies were sequentially sequenced with E44D (FIG. 4A), M184V (FIG. 4B) and L90M (FIG. 4C) primers. The mutation sites were extended with Cy5-dCTP (mutant) and Cy3-dATP (WT) for the E44D site, Cy5-dGTP (mutant) and Cy3-dATP (WT) for the M184 site, and Cy3-dATP (mutant) and Cy5-dUTP (WT) for the L90M site. A small area from each image was shown for better viewing. One representative polony from each clone is indicated with the numbered arrow. The color patterns for E44D, M184M, L90M and WT clones in three images are red-green-red (1), green-red-red (2) and green-green-green (3), and green-green-red (4), respectively.

FIGS. 5A-5C. Determination of primary drug resistance in the same viral genome by sequential PASS assays. WEAU.wt clone was used as the template in PASS assays and the polonies were sequenced using Cy3 or Cy5 labeled dNTP. The same polonies were sequentially probed with different sequencing primers for 22 primary drug-resistance mutation sites. Each sequencing primer was removed by denaturation in formamide at 45° C. before a new primer was annealed to the template in the same polonies. Results are grouped according to the drug classes. The numbers at the tope of the images indicate the order of the PASS assays. Each mutation site is indicated at the bottom of the image.

FIGS. 6A-6G. Detection of single base mutations among diverse HIV-1 subtypes and recombinants. Twenty-seven HIV-1 subtype or recombinant molecular clones were analyzed by PASS. They are two subtype As, eight subtype Bs, five subtype Cs, three subtype Ds, one subtype F, two subtype Gs, one subtype H, two CRF01_AEs, one CRF04_cpx and two other recombinants. The polonies were sequenced with E44D primer using Cy3-dCTP.

FIGS. 7A and 7B. Determination of sensitivity of the PASS assay by serial dilution of target molecules. WEAU.wt molecules (100,000, 10,000, 1,000, 100 and 10) were embedded in each gel (FIG. 7A). The actual genome numbers detected in the PASS assay are shown in parenthesis. After PCR amplification, the polonies were sequenced with E44D primer using Cy5-dATP. When 10,000 or more molecules were used for analysis, many spots tended to fuse to each other and were difficult to count, while the spots were easily and accurately countable when less than 10,000 molecules were used. When one or no copy was used in the assay, no polonies were detected (data not shown). The arrows indicate two polonies detected in the 10-molecule image. The experiments were repeated six times. The linear regression analysis were performed and plotted (FIG. 7B). The error bars stand for mean±SD. The spot numbers could not be reliably counted when 100,000 or more molecules were analyzed. Therefore, they were not included for estimation of assay sensitivity. Since the acrydite-modified primers were immobilized in the acrylamide gel and the PCR was carried out on a solid phase condition within the acrylamide gel, it was not surprising that not every expected molecule was detected in the PASS assay.

FIGS. 8A and 8B. Detection of multiple drug resistance mutations in a patient plasma sample. (FIG. 8A) Plasma samples from patients who were never treated before (naive), treated before but was not on any current therapy (experienced), or failed the on-going treatment regimen (failure) were analyzed by PASS. The RNA was extracted from 140 μl of plasma and cDNA equivalent to 14 μl plasma was used for each PASS assay. The M184V mutation site was determined with Cy5-dGTP (WT, red) and Cy3-dATP (mutant, green). The arrows indicate the minor resistance or WT viruses. (FIG. 8B) Twelve drug-resistance mutation sites were analyzed by PASS for the plasma sample from a treatment failure patient 200372. The same polonies were sequentially probed with 12 different sequencing primers. Each sequencing primer was removed by denaturation in formamide at 45° C. before a new primer was annealed to the same template. No drug resistance mutations were detected at four PI mutation sites (M46I, G48V, D30N and 184V), three RT sites associated with nucleoside resistance (V118I, E44D and K65R), and two RT sites associated with resistance to NNRTI (V106A and K103N). However, resistance was detected at two PI resistant mutation sites (L90M and V82A) as well as at M184V in RT. No viruses were found to be wild type at all mutation sites. This patient had previously been on other regimens including protease inhibitor regimens, likely explaining the presence of the PI mutations. Results are grouped according to the drug classes. Each mutation site is indicated at the bottom of the image.

FIGS. 9A-9D. Detection of drug-resistance mutations in patient plasma samples by PASS. The RNA was extracted from 140 μl of plasma and cDNA equivalent to 14 μl of plasma was used for each PASS assay. Twelve drug-resistance mutation sites were analyzed by PASS for the plasma sample from a treatment failure patient 200369. The same gel was sequentially probed with 12 different sequencing primers. Images containing resistance mutations (L100I, K103N and M184V) and one representative WT image at K65R position are shown. The percentages of minor resistance mutation populations are indicated at the top of the image. Each mutation site is indicated at the bottom of the image.

FIG. 10. Distribution of mutations.

### DETAILED DESCRIPTION OF THE INVENTION

The present invention relates to a parallel allele-specific sequencing (PASS) method for detecting mutations (e.g., MDR mutations or cancer associated mutations) in individual genomes (e.g., viral or cancer cell genomes). The method involves application of the polony technique (Mitra et al, Proc. Natl. Acad. Sci. USA 100:5926-5931 (2003); Zhu et al, Science 301:836-838 (2003)). The sensitivity of the instant method is such that it can be used to detect mutations when mutated genome numbers are low (e.g., when as few as six mutated genome copies are present) or when mutated genomes represent only a minor proportion (e.g., as low as about 0.01%) of the total genome copies present in a sample. For example, hundreds of thousands of viral genomes can be simultaneously analyzed in accordance with the instant invention and primary mutations in individual molecules can be determined by sequential interrogation of multiple mutation sites of the same molecules.

In a preferred embodiment, the present invention relates to a method of detecting a mutation or nucleotide modification in a multiplicity of nucleic acid templates (e.g., single or double stranded DNA) (e.g., from drug resistant pathogens or mutated cancer genomes) comprising:

i) embedding (e.g., casting) a multiplicity of nucleic acid templates and modified 3′ amplification primers or modified 5′ amplification primers in a semi-solid support under conditions such that the modified amplification primers become immobilized in (for example, covalently attached to) the semi-solid support;
ii) contacting (for example, overlaying) the semi-solid support resulting from step (i) with PCR components (e.g., polymerase, dNTPs, unmodified 5′ or 3′ amplification primers and buffer);
iii) treating the composition resulting from step (ii) under conditions such that the modified and/or unmodified amplification primers hybridize to the nucleic acid templates to form primed templates and effecting PCR amplification of the primed templates in the semi-solid support, wherein double-stranded products of the amplification localize around the templates;
iv) denaturing the double-stranded amplification products and removing (e.g., by washing) resulting single strands that are not immobilized in the semi-solid support;
v) hybridizing sequencing primers to immobilized single stands resulting from step (vi) and effecting extension of the hybridized sequencing primers in the presence of at least two nucleotides bearing different detectable labels, wherein one of the nucleotides is incorporated into the extension product when the immobilized single strand bears the mutation or nucleotide modification and the other of the nucleotides is incorporated into the extension product when the immobilized single strand does not bear the mutation or modified nucleotide (e.g., one of the labeled nucleotides is complementary to the mutated or modified nucleotide and the other of the labeled nucleotides is complementary to the nucleotide present in the wild type); and
vi) detecting the extension products of the sequencing primers resulting from step (v) whereby the percentage of extension products of the sequencing primers (and thus the percentage of templates) bearing the mutated or modified nucleotide can be determined.

In accordance with the present method, viral RNA or DNA can be extracted, for example, from a virus-containing biological sample (tissue or fluid (e.g., plasma, saliva or blood cell sample)) from a patient using, for example, standard protocols. Extracted RNA can be reverse-transcribed and the resulting cDNA isolated, e.g., using standard protocols. The cDNA or directly extracted DNA can then be subjected to PASS as described below.

Unmodified 5′ primers, modified 3′ primers, the DNA template and amplification reagents (e.g., PCR reagents) can be embedded in a semi-solid support (e.g., an acrylamide gel)—(alternatively, modified 5′ primers and unmodified 3′ primers can be used). Because the modified (e.g., acrydited) primers can be incorporated into the support during polymerization and become immobilized, the amplified products (e.g., PCR products) accumulate around the cDNA templates and form individual spots (polonies) at the amplification sites. Following amplification, the double-stranded DNA is denatured (e.g., using standard protocols, such as 70% of formamide) and the free DNA strand is removed (e.g., washed off). Only the anchored DNA strand remains associated with the support. Sequencing primers can then be annealed to the single-stranded templates and extended with labeled (e.g., fluorescent labeled) wild type or resistance bases. The supports can then be scanned to acquire images. When the primers are extended with, for example, fluorescence-labeled nucleotides, the base identity at the mutation site can be determined by the nucleotide and ultimately the fluorophore (or other label) incorporated. While in the Example below only two fluorescent dyes were detectable for WT and mutant bases, due to the instrument used, addition of a blue laser would permit detection of all four possible bases labeled with different fluorescent dyes (Shendure et al, Science 309:1728-1732 (2005)). As described in the Example that follows, one sequencing primer can be removed following denaturation and different sequencing primers can then be used sequentially until all desired mutations are determined.

The method of the present invention can be, for example, 200˜2,000 times more sensitive than convention genotypic and phenotypic assays (in terms of detecting minor resistance viral populations) (e.g., 0.1%˜0.01% vs. 20%). Thus, it can predict treatment outcomes more accurately. Since the actual number of viral genomes is determined with the instant PASS assay, the total number of polonies in each assay can also be used for viral load estimation, thereby enabling physicians to evaluate drug treatment efficacy and prognosis.

In the present method, viral cDNA or viral DNA molecules are directly introduced into the support (e.g., the semi-solid such as acrylamide gel) and amplification is effected at a single molecular level. Thus, artifact sequences generated through recombination or resampling during PCR are completed eliminated.

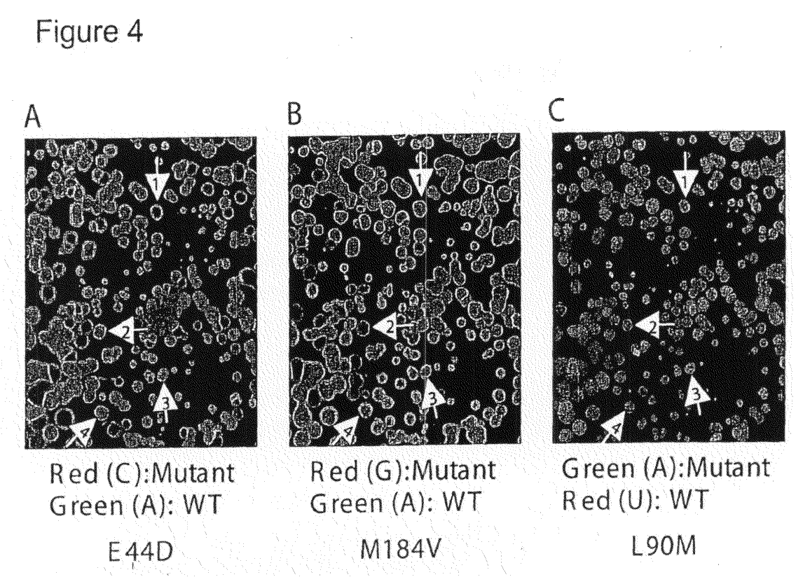

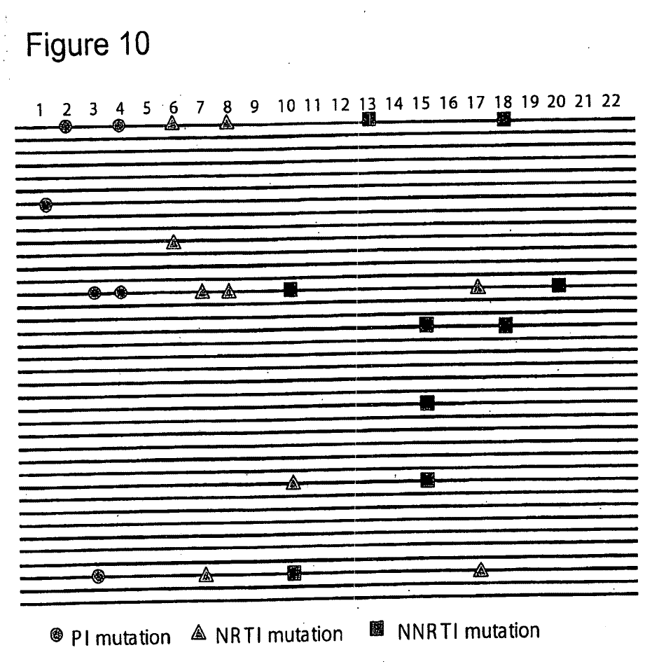

After determining all drug resistance mutations on each individual viral genome, the resistance mutations present in each viral genome will be known as will the number of mutations each viral genome carries, and the percentage of viral genomes containing resistance mutations (among the hundreds of thousands of viral genomes in each assay). Since all resistance mutations are obtained on a single molecule, linkage analysis (FIGS. 4 and 10) can be performed with the data obtained to determine MDR mechanisms and roles of recombination between single or simple MDR viruses in drug resistance by analyzing sequential samples from the patients.

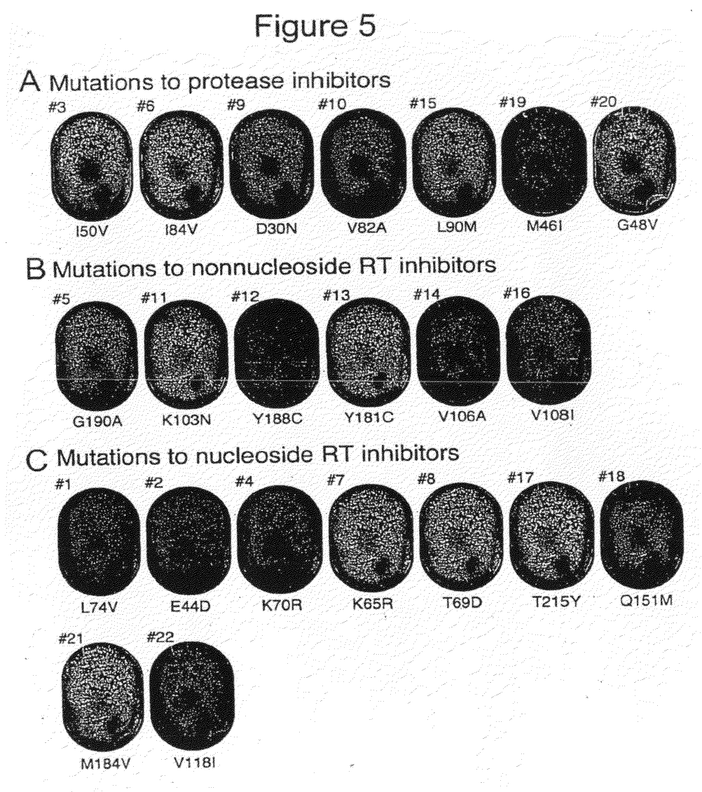

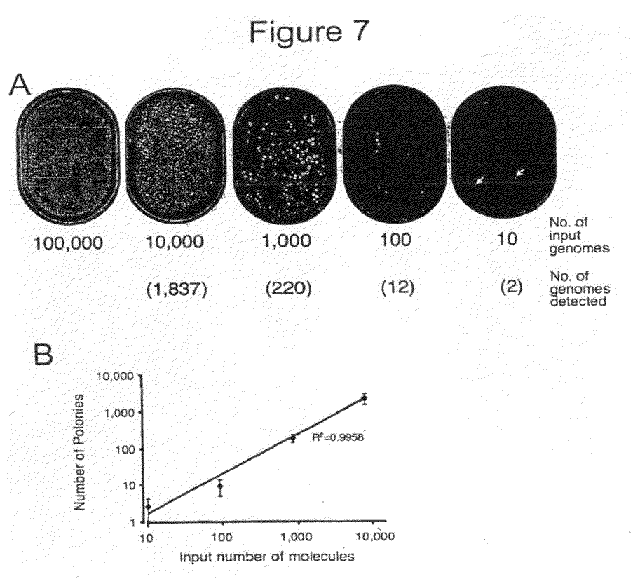

Viral loads are about 10⁵ RNA copies/ml on average in HIV-infected patients. If, for example, 140 μl equivalent of plasma (280 μl plasma for RNA extraction and half of that for PASS assay) is used, approximately 14,000 RNA copies are present in each PASS assay. With an 16% detection rate in the PASS assay (FIG. 5), 2,520 polonies will be detectable per assay. Therefore, the PASS assay is well suited for analyzing most of the patient plasma sample. Even when the viral is load is as high as 106 RNA copies/ml, detection of drug resistance mutations among 25,200 viral copies in one assay can be carried out since the assay can detect 1-10 in 50,000 molecules (FIG. 7). When viral load is too low, viruses can be concentrated, for example, from a large volume of plasma (e.g., by ultracentrifugation) for use in the PASS assay.

While the instant invention is described in detail in the Example that follows with reference to HIV, the PASS assay can also be used for detection of the presence of infectious agents (e.g., viral or bacterial) in a biological sample and/or for detection of drug resistance mutations in HBV, HCV or other infectious agents (e.g. Mycobacterium tuberculosis (TB)). Further, the PASS assay can be used to detect CTL and neutralizing antibody escape mutations.

In addition to the above, the present invention also has application in detection of mutations or methylated nucleosides in specimens from cancer patients. Gene-expression profiling can be useful in classifying tumors and determining prognosis in cancer patients (Hoheisel, Nat. Rev. Genet. 7:200-210 (2006); Schena et al, Science 270:467-470 (1995); Endoh et al, J. Clin. Oncol. 22:811-819 (2004); Lossos et al, N. Engl. J. Med. 350:1828-1837 (2004); Chen et al, N. Engl. J. Med. 356:11-20 (2007)). Certain mutations in host genes are responsible for development of cancers. To date, many mutations have been correlated with uncontrolled cell growth that can lead to tumor formation (for example, mutations associated with breast cancer have been reported). It has also been reported that the methylation of certain genes or promoters can significantly effect progress of cancers. Tumor cells carrying cancer related mutations or methylated nucleosides constitute only a portion of the total cell population in a biopsy tissue; a high percentage of cancer cells can indicate a more aggressive malignancy or a poor prognosis. Thus, it is crucial to be able to detect low percentages of mutations or methylated nucleosides in heterogeneous cancer samples. Recently, using the massively parallel picoliter reactor sequencing technology, 0.2% of mutations were detected in epidermal growth factor receptor gene (EGFR) in non-small-cell lung cancer (Thomas et al, Nat. Med. 12:852-855 (2006)). Such an assay can provide a benefit in selecting primary therapy and targeted inhibitors for relapsed patients. With a sensitivity of 0.01% for rare mutations, the PASS assay described herein can more effectively detect lower frequency of mutations or methylated nucleosides in heterogeneous cancer specimens. Biopsy tissue, blood or discharged materials (e.g., urine, feces or mucous or serous secretion (saliva), etc.) from individuals can be used for extraction of genomic DNA. The DNA can then be digested into smaller fragments for detection of cancer-related mutations or modified by sodium bisulfite treatment for detection of methylated nucleosides (Fisher et al, Lung Cancer Epub ahead of print Dec. 28, 2006); Tomii et al, Int. J. Cancer 120:566-573 (2007)). As described herein for standard PASS assay, the treated DNA can be embedded into the polyacrylamide gel and PASS analysis performed to detect rare cancer related mutations or methylated nucleosides. The method can be used for diagnosis and prognosis of cancers and for selection of primary therapy for better patient care.

Certain aspects of the invention are described in greater detail in the non-limiting Example that follows (see also polony amplification details in U.S. Pat. Nos. 6,432,360, 6,485,944, and 6,511,803, and U.S. Appln. Nos. 20030207265, 20030124594, 20020127552, 20060014167, 20020120127, 20030215856 and 20020120126). (See also Butz et al, BMC Biotechnology 3:11 (2003), Mitra and Church, Nucleic Acids Research 27(24):e34 (1999), U.S. Pat. No. 5,958,698, U.S. Pat. No. 5,616,478, Samatov et al, Nucleic Acids Research 33(17):e145 (2005), Samatov et al, Analytical Biochemistry 356:300-302 (2006), Chetverina et al, BioTechniques 33:150-156 (2002), Chetverina et al, Analytical Biochemistry 334:376-381 (2004).)

### EXAMPLE

### Experimental Details

Generation of drug resistance mutation clones. The partial pol gene containing most drug-resistance mutations was amplified from a near full-length HIV-1 clone WEAU.A1. E44D, M90L and M184V mutations were introduced by the overlapping PCR method. The wild type (WEAU.wt) and mutant PCR products (WEAU.E44D, WEAU.M90L and WEAU.M184M) were directly cloned into pSTBlue vector (Novagen, Madison, Wis.)). The drug-resistance mutations were confirmed by sequencing. For spiking experiments, the mutation and WT clones were mixed at various ratios (over 4 logs). The concentrations of plasmid DNAs were determined with a NanoDrop Spectrophotometer and the number of molecules in each DNA sample was calculated based on the DNA concentration and the plasmid length (bp).

Slide treatment. Teflon coated glass slides (Erie scientific, Portsmouth, N.H.) were exposed to UV light in a PCR hood for 15 min and subsequently treated with binding solution (0.02% glacial acetic acid and 0.4% bind-saline (Amersham Biosciences, Piscataway, N.J.)) for 1 hr. Slides were then rinsed three times with H₂O, twice with ethanol and dried in the PCR hood for 40 minutes. The treated slides were stored in a vacuum desiccator.

Gel casting. Slides and coverslips were exposed to UV light in a PCR hood for 15 minutes. The oval shaped space in the slide was filled with 20 μl of the gel mix containing: 1 μM acrydite-modified reverse primer, template (1-18 μl), 0.3% diallyltartramide, 5% rhinohide, 0.1% APS, 0.1% TEMED, 0.2% BSA in a 6% acrylamide gel. The gel mix was allowed to polymerize for 30 min. The coverslips were then removed with a razor blade. The slides were washed in 0:05% Tween 20 solution for 15 min and dried completely.

In-gel PCR amplification. The diffusion mixture of PCR reagents contained 1 μM forward primer, 0.1% Tween 20, 0.2% BSA, 1×PCR Buffer, 250 μM dNTP mix, 3.3 units of Jumpstart Taq DNA polymerase (Sigma, St. Louis, Mo.) and H₂O (up to 20 μl). Once the mixture was added to the center of the gel, the gel was immediately covered with a coverslip and kept at room temperature for 10 minutes. The gel with the coverslip was sealed with a SecurSeal chamber (Grace Bio-Labs, Bend, Oreg.). Each chamber was filled with 640 μl of mineral oil. A 1.1 kb pol gene fragment containing the protease and the partial RT genes was amplified with PAF1 5′-CTTTGGCAACGACCCCTCGTCACA-3′ (2265-2288, HXB2) and PAR2 5′-CCTGCATAAATCTGACTTGCCCAATTCAA-3′ (3367-3339). The PCR product contains all major resistance mutations to nucleoside RT inhibitors (NRTI), nonnucleoside RT inhibitors (NNRTI), and protease inhibitors (PI). The polony PCR was carried out with a PTC-200 Thermal Cycler (MJ Research, Hercules, Calif.) using the following conditions: 94° C. for 3 min; 65 cycles of 94° C. for 30 sec, 56° C. for 45 seconds, and 72° C. for 3 minutes; 72° C. for 6 minutes.

Single base extension (SBE). After PCR amplification, the slides were soaked in a Coplin jar with hexane for 15 minutes and washed with Wash 1E (10 mM Tris-HCl pH7.5, 50 mM KCl, 2 mM EDTA pH8.0 and 0.01% Triton X-100) four times. The slides were then denatured in formamide denaturation solution (70% formamide and 1×SSC) for 15 minutes. After washing once with water and twice with Wash 1E, a FrameSeal chamber (Bio-Rad, Hercules, Calif.) was applied to each slide and 125 μl of the annealing mix (6×SSPE, 0.01% Triton X-100 and 75 μl of 100 μM sequencing primer) was added to each slide. Hybridization of the sequencing primer was carried out in the PTC-200 Thermal Cycler at the following condition: 94° C. for 6 minutes and 53° C. for 20 minutes. After the primer was annealed to the template, the slides were washed twice in Wash 1E and then equilibrated with Klenow buffer (50 mM Tris-HCl pH7.4, 5 mM MgCl, and 0.01% Triton X-100) at room temperature for three minutes. Forty-five microliters of extension mixture (0.5 μM Cy5 labeled deoxynucleotide {WT or mutant}, 0.5 μM Cy3 labeled different deoxynucleotide {mutant or WT}, 1% Single-Stranded DNA Binding Protein {USB corporation, Cleveland, Ohio} and 10 units of Klenow in Klenow buffer) were added to each gel. The slides were incubated at room temperature for three minutes, washed twice with Wash 1E, and scanned using ScanArray Express (PerkinElmer, Wellesley, Mass.).

Sequencing additional drug resistance mutations. To determine primary drug-resistance mutations in individual protease/RT amplicons, the polony slides were treated with formamide denaturation solution at 45° C. for 15 min, followed by the rinse with H₂O and two rinses with Wash 1E. The slides were scanned to ensure complete removal of sequencing primer from previous SBE, they were then annealed with a different sequencing primer. The SBE was repeated until all desired mutations were determined (see Table 1 and FIG. 5).

TABLE 1
Primers used for the PASS assay

| | |
|---|---|
| Primer | Primer sequences |

PCR amplification primers

| | |
|---|---|
| PAF1 | CTTTGGCAACGACCCCTCGTCACA |
| PAF2 | CCAAAAATTGCAGGGCCCCTAGGAA |
| PAR1-Acr | CCCACTAACTTCTGTATGTCATTGACAGTCCA |
| PAR2-Acr | CCTGCATAAATCTGACTTGCCCAATTCAA |

Sequencing primers in the protease gene

| | |
|---|---|
| D30Nga | GAAGCTCTATTAGATACAGGAGCAGAT |
| M46Iga | AATTTGCCAGGAAGATGGAAACCAAAAAT |
| G48Vgt | CCAGGAAGATGGAAACCAAAAATGATAG |
| I50Vag | TGGAAACCAAAAATGATAGGGGGA |
| V82Atc | GGTACAGTATTAGTAGGACCTAGACCTG |
| I84Vag | CAGTATTAGTAGGACCTACACCTGTCAAC |
| L90Mta | ACCTGTCAACATAATTGGAAGAAATCTG |

Sequencing primers in the reverse
transcriptase gene

| | |
|---|---|
| M41La-t/c | AAAGCATTAGTAGAAATTTGTACAGAA |
| E44Dac | GTAGAAATTTGTACAGAAATGGAAAAGGA |
| K65Rag | CAATACTCCAGTATTTGCCATAAAGA |
| D67Nga | ACTCCAGTATTTGCCATAAAGAAAAAA |
| T69Dac.ga | GTATTTGCCATAAAGAAAAAAGACAGT |
| K70Rag | TTGCCATAAAGTAAAAAGACAGTACTA |
| L74Vtg | AAAAAGACAGTACTAAATGGAGAAAA |
| L100Ita | GTTCAATTAGGAATACCACATCCCGCAGGG |
| K103Nac | ACATCCCGCAGGGTTAAAAAAGAA |
| V106Atc | GCAGGGTTAAAAAAGAAAAAATCAG |
| V108Iga | AGGGTTAAAAAAGAAAAAATCAGTAACA |
| V118Iga | GGATGTGGGTGATGCATATTTTTCA |
| Q151Mca.at | ATTAGATATCAGTACAATGTGCTTCCA |
| Y181Cag | AGAAAACAAAATCCAGACATAGTTATCT |
| M184Vag | CAAAATCCAGACATAGTTATCTATCAATAC |
| Y188CLHta.t/c.g/a | ATAGTTATCTATCAATACATGGATGATTTG |
| G190Agc | TCTATCAATACATGGATGATTTGTATGTAG |
| L210Wtg | AAAAATAGAGGAGCTGAGACAACCATCTGT |
| T215Y/Fac.tt/a | CAACATCTGTTGAGGTGGGGATTT |
| K219Eag | TTGAGGTGGGGACTTACCACACCAGAC |
| P225Hca | CCAGACAAAAAACATCAGAAAGAAC |

Viral RNA extraction and reverse transcription. Viral RNA (vRNA) was extracted from 280 μl of plasma samples from HIV-1 infected individuals using QIAamp Viral RNA Mini Kit (Qiagen Inc., Valencia, Calif.). The vRNA was eluted into 60 μl of AVE buffer and 17 μl of vRNA was used for cDNA synthesis using Superscript III (Invitrogen Corp., Carlsbad, Calif.) and primer RTuni1 5′-CCAATCCCCCCTTTTCTTTTA AAATTGTG-3′ in a 40 μl volume. About half of the cDNA (18 μl) was used for one PASS assay.

### Results

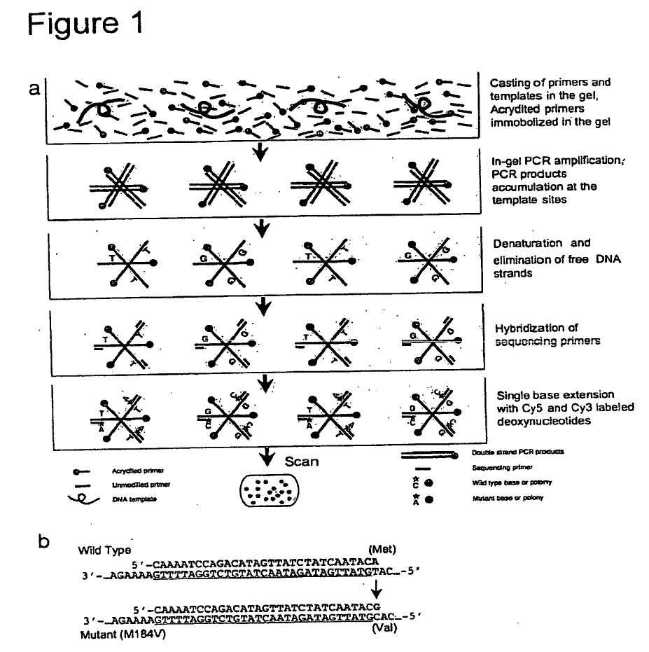

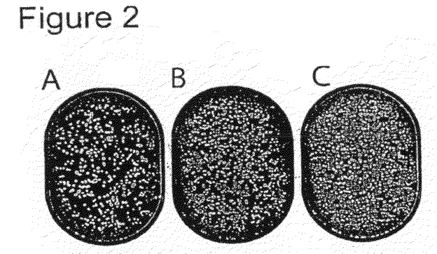

Parallel detection of single drug resistance mutations. Recently a polymerase colony (polony) technology has been developed to detect single-base mutations at a single molecule level in a solid phase (Mitra et al, Proc Natl Acad Sci USA 100:5926-31 (2003), Mitra et al, Anal Biochem 320:55-65 (2003)). The method has been used for profiling combinatorial alternative pre-mRNA splicing and parallel sequencing (Shendure et al, Science 309:1728-32 (2005), Zhu et al, Science 301:836-8 (2003)). To detect minor resistant viral populations, a parallel allele-specific sequencing (PASS) method was developed by applying the polony technique to simultaneously analyze hundreds of thousands of viral genomes in a single assay. A 1.1 kb pol gene fragment containing the protease and partial reverse transcriptase (RT) genes was amplified. The amplicon covers all major resistance mutations to RT and protease. Polony amplification was carried out in an acrylamide gel. Because the acrydite-modified primer becomes immobilized by covalently incorporating into polyacrylamide gels during polymerization, the PCR products accumulate around the DNA templates and form individual spots (polonies) at the amplification sites (FIG. 1A). After amplification, the negative DNA strands were hybridized with complementary sequencing primers, the 3′ end of the primers being juxtaposed to the resistance mutation base. When the primer was extended by single-base extension (SBE), the base-identity at the mutation site was determined by incorporated nucleotides that were labeled with different fluorophores (FIG. 1B). Images were acquired with a micro array scanner for analysis of WT and mutation populations (FIG. 2).

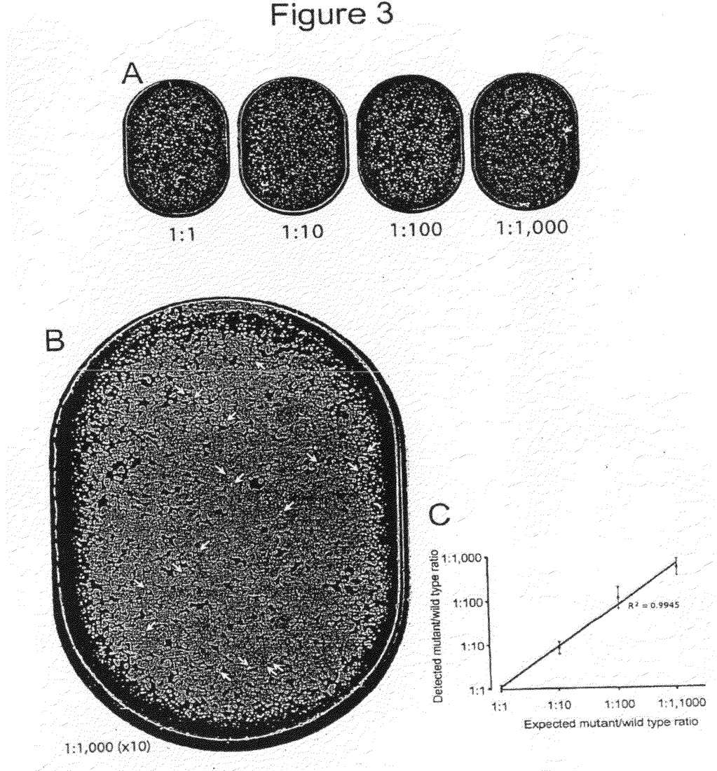

To determine the sensitivity of the PASS assay, WT molecules (WEAU-wt) were spiked with mutant molecules (WEAU-E44D) at various ratios (1:1, 1:10, 1:100, and 1:1,000). The mixtures were then detected with the E44D primer. Mutations present at 0.1% or higher were easily detected (FIG. 3A). When the viral genome input was increased to 10,000 at the same 1:1,000 mutant/WT ratio, resistance mutations were also readily detected, although a proportion of WT molecules were fused to one another and could not be accurately counted (FIG. 3B). Therefore, the assay sensitivity for detection of minor resistance mutations may be as high as about 0.01%. Linear regression analysis revealed a good linear correlation (R²=0.9945) between detected and expected mutant/WT ratios (FIG. 3C).

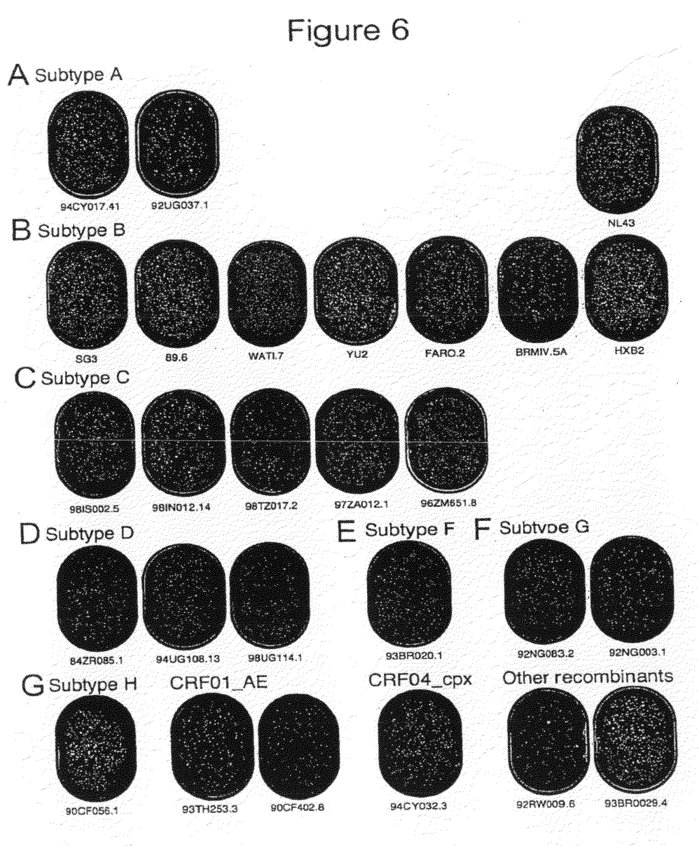

Twenty two primary mutations were detected on individual molecules through multiple SBEs, suggesting that the PASS assay can be used to detect most primary mutations and potentially other secondary mutations for linkage analysis when more sequencing cycles are performed (FIG. 5). Also tested were 27 viral genomes from many subtypes and recombinant forms (2 As, 8 Bs, 5 Cs, 3 Ds, 1 F, 2 Gs, 1H, 2 CRF01_AEs, 1 CRF04_cpx and 2 other recombinants). All tested subtypes and recombinants were comparably amplified, suggesting that the PASS assay can be used for most genetic subtypes and recombinants (FIG. 6). The sensitivity of the assay for detection of both WT and mutant populations was about 6 copies of viral genomes. Linear regression analysis showed a good linear detection correlation (R²=0.9958) between 10 and 10,000 copies of viral genomes (FIG. 7). No mutant bases were detected when more than 200,000 WT molecules were analyzed, suggesting that the mutation rate introduced by Taq polymerase was lower than 5×10⁻⁶ in the current experimental conditions. Therefore, low frequency mutations detected by PASS will not likely represent PCR errors.

To detect different resistance mutations in each molecule present in a viral population, three single drug-resistance clones were generated (WEAU-E44D, WEAU-L90M and WEAU-M 184V) and they were mixed with the WT clone (WEAU-wt) at a 1:1:1:1 ratio. The polonies were then sequentially sequenced with E44D, M184V and L90M primers. As expected, ˜25% of the polonies contained the resistance mutation and the other 75% of polonies were wild type in each reaction (FIG. 4A-4C). When polonies were analyzed from all three images, the WT clones showed as a green-green-red pattern, while E44D clones showed as red-green-red, M184V as green-red-red and L90M as green-green-green. This result demonstrates that the PASS assay can accurately determine the presence of different resistance mutations in each viral genome for linkage analysis when a large number of viral genomes are analyzed in parallel.

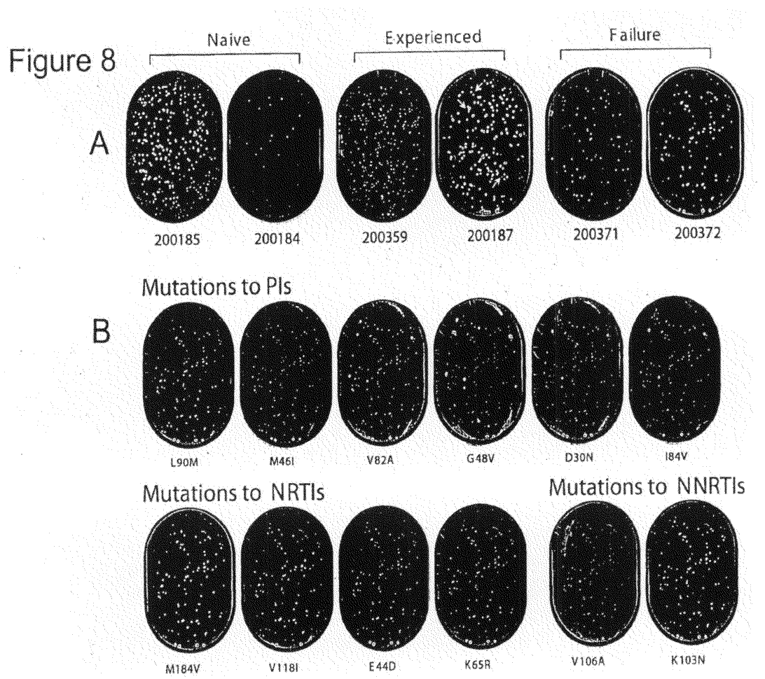

To determine resistance populations in samples from HIV-infected patients using the PASS assay, 13 plasma samples were analyzed from three different patient groups: patients who were never treated before (naïve), patients previously treated but not currently on antiretroviral therapy (experienced), and patients failing a current treatment regimen (failure). The M184V mutation was first determined and no mutations were detected in treatment-naïve patients, minor resistant populations (<2%) in two of six previously treated patients, and major resistance populations in treatment failure patients (36%-100%) (FIG. 8 and Table 2).

TABLE 2
Detection of the A to G drug resistance mutation in the M184V codon

| | | | | Number of viral genome | | |
|---|---|---|---|---|---|---|
| | | Viral load | | Number of | Number of | % of drug |
| | Patient | (RNA | Drug used in | genomes with | WT | resistance |
| Treatment | ID | copies/ml) | treatment | mutations | genomes | genomes |
| Naïve | 200184 | 15,024 | None | 0 | 27 | 0 |
| | 200185 | 13,200 | None | 0 | 333 | 0 |
| | 200189 | 5,479 | None | 0 | 4 | 0 |
| | 200192 | 23,518 | None | 0 | 10 | 0 |
| Experienced | 200356 | 62,242 | AZT, 3TC | 0 | 29 | 0 |
| | 200357 | 33,677 | AZT, 3TC, | 0 | 120 | 0 |
| | | | IDV | | | |
| | 200358 | 5,116 | LPV/R, AZT | 0 | 5 | 0 |
| | 200359 | 476,944 | AZT, 3TC, | 0 | 311 | 0 |
| | | | IDV | | | |
| | 200362 | 127,895 | 3TC, ABC, | 1 | 60 | 1.7 |
| | | | AMP, NVP, | | | |
| | | | RTV | | | |
| | 200187 | 104,535 | AZT, 3TC, | 3 | 219 | 1.4 |
| | | | SQV, ddC | | | |
| Failure | 200369 | 189,146 | ddl, d4T, | 491 | 863 | 36.3 |
| | | | SQV and | | | |
| | | | previous | | | |
| | | | other drugs | | | |
| | 200371 | 60,476 | AZT, 3TC, | 85 | 0 | 100 |
| | | | NVP | | | |
| | 200372 | 234,528 | d4T, 3TC, | 123 | 0 | 100 |
| | | | LPR [symbol] | | | |

[symbol]

indicates data missing or illegible when filed

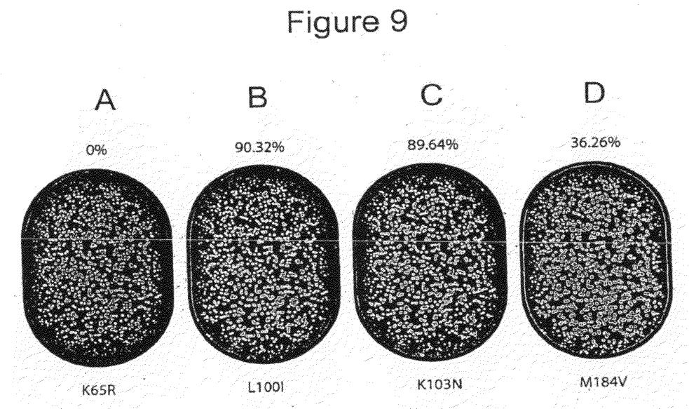

To determine if other resistance mutations were present among studied viral populations, 12 potential resistance mutation sites were analyzed in seven patient samples representing the three groups (Table 3). Additional resistance mutations were detected in one experienced patient and all three failure patients. L90M and V82A mutations were detected in patient 200372, and K70R and T215Y mutations were detected in patient 200371 (Table 3 and FIG. 7). No WT viruses were found in either patient. In two other patients, both resistant and WT viruses were detected at multiple resistance mutation sites (FIG. 9). This made it possible to perform detailed linkage analysis. In treatment failure patient 200369, the L1001 mutation was present in 90.32% of the viruses and the K103N mutation was present in 89.64% of the viruses. The 36.26% M184V mutant viruses were not detected in the standard genotypic assay but readily detected in the PASS assay (FIG. 9). Linkage analysis showed that more than half of the viruses (52.14%) carried both L100I and K103N mutations, and 35.10% of the viruses had three resistance mutations (L100I, K103M and M184V). Other viruses containing single or dual resistance mutations were present as minor populations (L100I 0.66%, K103N 0.07%, M184V 0.07% and M100I/M184V 0.07%) (Table 3). The previously treated patient 200362 also showed a similar resistance mutation linkage profile. Although the vast majority of viruses contained one or more resistance mutations, both patients still harbored minor WT virus populations (1.67% and 9.22%).

TABLE 3
Linkage analysis of drug-resistance mutations
in individual viral genomes

| | | No. of | | | |
|---|---|---|---|---|---|
| | | mutation | | | Per- |
| Treat- | | sites | Pattern of | No. of | centage |
| ment | Patient | analyzed | linked mutations | genomes | (%) |
| Naïve | 200184 | 12 | wild type | 27 | 100 |
| Expe- | 200187 | 12 | wild type | 219 | 98.65 |
| rienced | | | M184V | 3 | 1.35 |
| | 200359 | 12 | wild type | 311 | 100 |
| | 200362 | 12 | wild type | 1 | 1.67 |
| | | | Y181C | 4 | 6.67 |
| | | | K70R | 42 | 70.00 |
| | | | V118I + Y181C | 1 | 1.67 |
| | | | K70R + Y181C | 2 | 3.33 |
| | | | K70R + V118I + | 1 | 1.67 |
| | | | Y181C + M184V | | |
| | | | others | 9 | 15.00 |
| Failure | 200369 | 12 | wild type | 125 | 9.22 |
| | | | L100I | 9 | 0.66 |
| | | | K103N | 1 | 0.07 |
| | | | M184V | 1 | 0.07 |
| | | | L100I + M184V | 1 | 0.07 |
| | | | L100I + K103N | 707 | 52.14 |
| | | | L100I + K103N + | 476 | 35.10 |
| | | | M184V | | |
| | | | others | 36 | 2.65 |
| | 200371 | 9 | M184V + K70R + | 85 | 100 |
| | | | T215Y | | |
| | 200372 | 12 | L90M + V82A + | 123 | 100 |
| | | | M184V | | |

Consistent improvement in HIV treatment outcomes as a consequence of genotypic or phenotypic testing among patients failing antiretroviral treatment has not been confirmed (Johnson et al, Top. HIV Med. 13:125-131 (2005)). This may partially be due to the low sensitivity (presence of >20% mutant viruses) of the assays for detection of minor resistance populations in such patients (Petropoulos et al, Antimicrob. Agents Chemother. 44:920-928 (2000), Van Laethem et al, J. Acquir. Immune Defic. Syndr. 22:107-118 (1999)). Point mutation assays offer significantly greater sensitivity than either genotypic or phenotypic assays (Van Laethem et al, J. Acquir. Immune Defic. Syndr. 22:107-118 (1999)). Since drug-resistance mutations detected by these methods are population-based, none of them allow linkage analysis. Instead, clonal sequencing has been used to study minor resistance populations and linkage analysis of resistance mutations on each viral genome (Condra et al, Nature 374:569-571 (1995)). Although it takes a few days to analyze all primary mutations using the PASS assay, this approach is substantially more efficient, faster and more sensitive for detection of minor resistance populations than is clonal sequencing. While the PASS assay takes longer to complete than standard genotypic assays, it offers far better sensitivity for detection of minor resistance mutations and for linkage analysis. Although the clinical significance of minor resistance viral populations has not been fully determined, it is likely that such populations affect subsequent treatment outcomes significantly (Charpentier et al, J. Virol. 78:4234-4247 (2004), Metzner et al, J. Infect. Dis. 188:1433-1443 (2003), Winters et al, J. Virol. 72:5303-5306 (1998)). Because the PASS method is about 1,000 times more sensitive than either genotypic or phenotypic assay (0.01% vs. 20%) for detection of minor resistance viral populations, it may permit more accurate prediction of treatment outcomes. The PASS assay permits a detailed linkage analysis of multiple mutations, allowing study of the impact of different combinations of mutations for minor and major viral populations (Table 3). In addition to these clinical improvements, technical advantages of the PASS assay are notable. For example, in the PASS procedure viral cDNA molecules are directly embedded into polyacrylamide gel and PCR amplification is carried out at a single molecular level. Therefore, artifact sequences that are generated through recombination or resampling during conventional PCR are eliminated (Fang et al, J. Virol. Methods 76:139-148 (1998), Liu et al, Science 273:415-416 (1996)).

Summarizing, described above is a PASS assay that can detect minor resistance populations in HIV-infected individuals with high sensitivity, specificity and throughput. The approach applies the polony technique, which has been used for profiling combinatorial alternative pre-mRNA splicing, parallel detection of single base mutation and parallel sequencing (Zhu et al, Science 301:836-838 (2003), Merrit et al, Biotechnol. Bioeng. 92:519-531 (2005), Butz et al, BMC Genet. 5:3 (2004), Shendure et al, Science 309:1728-1732 (2005)). The new technology may also potentially be used to detect IV escape mutations in antigen determinants targeted by T and B cells, other human pathogens (like hepatitis B and C virus, TB, etc) and cancer related mutations or nucleoside modifications.

All documents and other information sources cited above are hereby incorporated in their entirety by reference.

What is claimed is:

1. A method of detecting, or determining the frequency of, a mutation or nucleotide modification in a multiplicity of nucleic acid templates comprising:
i) embedding a multiplicity of nucleic acid templates and modified 3′ amplification primers or modified 5′ amplification primers in a semi-solid support under conditions such that said modified amplification primers are immobilized in said semi-solid support;
ii) contacting said semi-solid support resulting from step (i) with a polymerase, deoxynucleotide triphosphates (dNTPs), and unmodified 5′ or 3′ amplification primers;
iii) treating the composition resulting from step (ii) under conditions such that said modified and/or unmodified amplification primers hybridize to said nucleic acid templates to form primed templates;
iv) effecting amplification of said primed templates in said semi-solid support, wherein double-stranded products of said amplification localize around said primed templates;
v) denaturing said double-stranded amplification products and removing resulting single strands not immobilized in said semi-solid support;
vi) hybridizing sequencing primers to said immobilized single stands resulting from step (v) and effecting extension of said hybridized sequencing primers in the presence of at least two nucleotides bearing different detectable labels to form extension products, wherein one of said nucleotides is incorporated into said extension product when said immobilized single strand bears said mutation or nucleotide modification and the other of said nucleotides is incorporated into said extension product when said immobilized single strand does not bear said mutation or modified nucleotide; and
vii) detecting the extension products of the sequencing primers resulting from step (vi), and determining the presence of, or the frequency of, said nucleic acid templates bearing said mutated or modified nucleotide.

2. The method according to claim 1 wherein said method is a method of determining the frequency of a mutation.

3. The method according to claim 1 wherein said multiplicity of nucleic acid templates comprise double-stranded DNA.

4. The method according to claim 1 wherein said multiplicity of nucleic acid templates are derived from a pathogen.

5. The method according to claim 4 wherein said pathogen is a viral or bacterial pathogen.

6. The method according to claim 5 wherein said pathogen is human immunodeficiency virus (HIV), hepatitis B virus (HBV), hepatitis C virus (HCV) or Mycobacterium tuberculosis (TB).

7. The method according to claim 5 wherein said pathogen is a drug resistant pathogen.

8. The method according to claim 1 wherein said multiplicity of nucleic acid templates are derived from genomes from cancer cells.

9. The method according to claim 8 wherein said cancer cells are human cancer cells.

10. The method according to claim 1 wherein said cancer cells are derived from a biopsy tissue, from blood or from a discharged material.

11. The method according to claim 10 wherein said discharged material comprises urine, feces or mucous or serous secretion.

12. The method according to claim 1 wherein said modified amplification primers are covalently attached to said semi-solid support.

13. The method according to claim 12 wherein said semi-solid support is an acrylamide gel.

14. The method according to claim 1 wherein unmodified 5′ primers and modified 3′ primers are embedded in said semi-solid support.

15. The method according to claim 1 wherein unmodified 3′ primers and modified 5′ primers are embedded in said semi-solid support.

16. The method according to claim 1 wherein said modified primers are acrydited primers.

17. The method according to claim 1 wherein said detectable labels are fluorescent labels.

18. A method of determining viral load in a patient comprising:
i) extracting viral DNA from a biological sample from said patient;
ii) embedding said DNA and modified 3′ amplification primers or modified 5′ amplification primers in a semi-solid support under conditions such that said modified amplification primers are immobilized in said semi-solid support;
iii) contacting said semi-solid support resulting from step (i) with a polymerase, deoxynucleotide triphosphates (dNTPs), and unmodified 5′ or 3′ amplification primers;
iv) treating the composition resulting from step (iii) under conditions such that said modified and/or unmodified amplification primers hybridize to said DNA to form primed templates,
v) effecting amplification of said primed templates in said semi-solid support, wherein double-stranded products of said amplification localize around said primed templates;
vi) denaturing said double-stranded amplification products and removing resulting single strands not immobilized in said semi-solid support;
vii) hybridizing sequencing primers to said immobilized single stands resulting from step (vi) and effecting extension of said hybridized sequencing primers in the presence of at least one nucleotide bearing a detectable label to form extension products; and
viii) detecting labeled extension products resulting from step (vii), and thereby determining said viral load.

19. The method according to claim 18 wherein, prior to step (ii), said DNA is sheared.

20. The method according to claim 19 wherein said sheared DNA is about 100 bases to about 1000 kb in length.

21. The method according to claim 20 wherein said sheared DNA is about 1 kb to about 100 kb in length.

22. A method of determining viral load in a patient comprising:
i) extracting viral RNA from a biological sample from said patient;
ii) reverse transcribing said RNA to produce cDNA and embedding said cDNA and modified 3′ amplification primers or modified 5′ amplification primers in a semi-solid support under conditions such that said modified amplification primers are immobilized in said semi-solid support;
iii) contacting said semi-solid support resulting from step (i) with a polymerase, deoxynucleotide triphosphates (dNTPs), and unmodified 5′ or 3′ amplification primers;
iv) treating the composition resulting from step (iii) under conditions such that said modified and/or unmodified amplification primers hybridize to said cDNA to form primed templates,
v) effecting amplification of said primed templates in said semi-solid support, wherein double-stranded products of said amplification localize around said primed templates;
vi) denaturing said double-stranded amplification products and removing resulting single strands not immobilized in said semi-solid support;
vii) hybridizing sequencing primers to said immobilized single stands resulting from step (vi) and effecting extension of said hybridized sequencing primers in the presence of at least one nucleotide bearing a detectable label to form labeled extension products; and
viii) detecting labeled extension products resulting from step (vii), and thereby determining said viral load.

23. A method of determining viral load in a patient comprising:
i) extracting viral DNA from a biological sample from said patient;
ii) embedding said DNA and modified 3′ amplification primers or modified 5′ amplification primers in a semi-solid support under conditions such that said modified amplification primers are immobilized in said semi-solid support;
iii) contacting said semi-solid support resulting from step (i) with a polymerase, deoxynucleotide triphosphates (dNTPs), and unmodified 5′ or 3′ amplification primers;
iv) treating the composition resulting from step (iii) under conditions such that said modified and/or unmodified amplification primers hybridize to said DNA to form primed templates,
v) effecting amplification of said primed templates in said semi-solid support, wherein double-stranded products of said amplification localize around said primed templates;
vi) denaturing said double-stranded amplification products and removing resulting single strands not immobilized in said semi-solid support;
vii) hybridizing a labeled probe specific for said virus to said immobilized single stands resulting from step (vi) to form a labeled duplex; and
viii) detecting the presence of said labeled duplex, and thereby determining said viral load.

24. The method according to claim 23 wherein, prior to step (ii), said DNA is sheared.

25. A method of determining viral load in a patient comprising:
i) extracting viral RNA from a biological sample from said patient;
ii) reverse transcribing said RNA to form cDNA and embedding said cDNA and modified 3′ amplification primers or modified 5′ amplification primers in a semi-solid support under conditions such that said modified amplification primers are immobilized in said semi-solid support;
iii) contacting said semi-solid support resulting from step (i) with a polymerase, deoxynucleotide triphosphates (dNTPs), and unmodified 5′ or 3′ amplification primers;
iv) treating the composition resulting from step (iii) under conditions such that said modified and/or unmodified amplification primers hybridize to said cDNA to form primed templates,
v) effecting amplification of said primed templates in said semi-solid support, wherein double-stranded products of said amplification localize around said primed templates;
vi) denaturing said double-stranded amplification products and removing resulting single strands not immobilized in said semi-solid support;
vii) hybridizing a labeled probe specific for said virus to said immobilized single stands resulting from step (vi) to form a labeled duplex; and
viii) detecting the presence of said labeled duplex, and thereby determining said viral load.

26. A method of detecting a mutation or nucleotide modification in a multiplicity of nucleic acid templates derived from DNA isolated from a biological sample comprising:
i) embedding said multiplicity of nucleic acid templates and modified 3′ amplification primers or modified 5′ amplification primers in a semi-solid support under conditions such that said modified amplification primers are immobilized in said semi-solid support;
ii) contacting said semi-solid support resulting from step (i) with a polymerase, deoxynucleotide triphosphates (dNTPs), and unmodified 5′ or 3′ amplification primers;
iii) treating the composition resulting from step (ii) under conditions such that said modified and/or unmodified amplification primers hybridize to said nucleic acid templates to form primed templates,
iv) effecting amplification of said primed templates in said semi-solid support, wherein double-stranded products of said amplification localize around said primed templates;
v) denaturing said double-stranded amplification products and removing resulting single strands not immobilized in said semi-solid support;
vi) hybridizing sequencing primers to said immobilized single stands resulting from step (v) and effecting extension of said hybridized sequencing primers in the presence of a nucleotide bearing a detectable label, wherein said nucleotide bearing said detectable label is incorporated into said extension product when said immobilized single strand bears said mutation or nucleotide modification so that a labeled extension product is formed; and
viii) detecting labeled extension products resulting from step (vii), and thereby determining the presence of said mutation or nucleotide modification.

27. The method according to claim 26 wherein said biological sample comprises DNA from cancer cells.
